(19) United States
(12) Patent Application Publication
(10) Pub. No.: US 2024/0299331 A1
(43) Pub. Date: Sep. 12, 2024

(54) PAIN TREATMENT FORMULATIONS AND METHODS OF USING THE SAME

(71) Applicant: Robert Pelletier, Dixfield, ME (US)

(72) Inventor: Robert Pelletier, Dixfield, ME (US)

(21) Appl. No.: 18/599,632

(22) Filed: Mar. 8, 2024

Related U.S. Application Data
(60) Provisional application No. 63/489,252, filed on Mar. 9, 2023.

(51) Int. Cl. A61K 31/245; A61K 9/00; A61K 31/165; A61P 29/02

(52) U.S. Cl. CPC A61K 31/245; A61K 9/0014; A61K 31/165; A61P 29/02

(57) ABSTRACT

Disclosed are pharmaceutically acceptable topical formulations having enhanced potency in the alleviation of pain. Such formulations can be useful in the treatment of conditions such as back, skeletal, and joint pain, where the pharmaceutically acceptable topical formulations are applied topically to the site of pain.

### CROSS REFERENCE TO RELATED APPLICATIONS

This application claims the benefit of and priority to U.S. Patent Application No. 63/489,252, filed on Mar. 9, 2023, the entire contents of which are incorporated by reference herein.

### BACKGROUND

Pain can function as a protective mechanism that allows healthy human beings and animals to avoid tissue damage and/or prevent further damage to injured tissue. However, there are many instances in which pain persists beyond its usefulness. Such unnecessary suffering from pain can impair a subject's physical mobility, mental performance, and even contribute to depression.

Substantial resources have been devoted over the years to researching the causes of various types of pain and to the development of medicine to attenuate pain experienced by a patient. Exemplary classes of common pain-relief medications include opioids, non-steroidal anti-inflammatory agents, corticosteroids, and centrally acting agents such as anti-depressants, anti-epileptics, pregabalin, and gabapentin.

A need continues to exist in the art for novel and more specific and/or potent formulations that are capable of repressing or ceasing painful conditions, such a joint pain. In addition, a need continues to exist in the medical arts for topically deliverable forms of such formulations.

### SUMMARY

The present disclosure includes pharmaceutically acceptable topical formulations that are useful in the treatment of pain. The topical formulations, such as a cream or a gel or a combination thereof, generally include benzocaine and capsaicin. In particular, the present disclosure provides a method of treating a pain condition such as muscle or joint pain, or skeletal pain, or spasm induced pain, for example, muscle spasm induced pain. Such methods can include topically administering to the patient a therapeutically effective amount of a disclosed pharmaceutically acceptable topical formulation.

### DETAILED DESCRIPTION

It has now been discovered that pharmaceutically acceptable topical formulations generally including benzocaine and capsaicin can be useful in the treatment of a variety of pain related conditions such a back pain, spinal or skeletal pain, joint pain, and muscle spasm induced pain. More specifically, in various embodiments, the pharmaceutically acceptable topical formulation includes benzocaine, in an amount from about 5% (w/w) to about 20% (w/w) based on the total weight of the pharmaceutically acceptable topical formulation; and capsaicin, in an amount from about 0.005% (w/w) to about 0.1% (w/w) based on the total weight of the pharmaceutically acceptable topical formulation, where the pharmaceutically acceptable topical formulation is for topical application.

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate.

The terms “individual,” “patient,” and “subject,” as used herein, are used interchangeably and include any animal, including mammals, preferably mice, rats, other rodents, rabbits, dogs, cats, swine, cattle, sheep, horses, or primates, and more preferably, humans. The formulations described in the disclosure can be administered to a mammal, such as a human, but can also be administered to other mammals such as an animal in need of veterinary treatment, for example, domestic animals (e.g., dogs, cats, and the like), farm animals (e.g., cows, sheep, pigs, horses, and the like) and laboratory animals (e.g., rats, mice, guinea pigs, and the like). The mammal treated in the methods described in the disclosure is preferably a mammal in which treatment, for example, of pain, is desired.

The term “treating,” as used herein, includes any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The phrase “therapeutically effective amount,” as used herein, refers to the amount of a formulation (e.g., a disclosed formulation) that will elicit the biological or medical response of a tissue, system, animal or human that is being sought by the researcher, veterinarian, medical doctor or other clinician. The compounds described in the disclosure can be administered in therapeutically effective amounts to treat pain. A therapeutically effective amount of a compound can be the quantity required to achieve a desired therapeutic and/or prophylactic effect, such as an amount which results in lessening of a symptom of a condition such as pain.

Unless defined otherwise, all technical and scientific terms used herein have the same meaning as commonly understood by one of ordinary skill in the art to which this disclosure pertains.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a formulation or a method described herein can be combined in a variety of ways without departing from the spirit and scope of the present disclosure, whether explicit or implicit herein. For example, where reference is made to a particular compound, that compound can be used in various embodiments of formulations of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise. As used herein, the term “about” refers to a ±10%, ±5%, ±3%, ±2%, ±1%, variation from the nominal value as appropriate for the variable and circumstances unless otherwise indicated or inferred.

Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

At various places in the present specification, substituents are disclosed in groups or in ranges. It is specifically intended that the description include each and every individual subcombination of the members of such groups and ranges. By way of example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

The present teachings generally provide a pharmaceutically acceptable topical formulation comprising benzocaine, where benzocaine is present in an amount from about 5% (w/w) to about 20% (w/w) based on the total weight of the pharmaceutically acceptable topical formulation; and capsaicin, where the capsaicin is present in an amount from about 0.005% (w/w) to about 0.1% (w/w) based on the total weight of the pharmaceutically acceptable topical formulation, and wherein the pharmaceutically acceptable topical formulation is for topical application.

In various embodiments, the pharmaceutically acceptable topical formulation includes a polyethylene glycol. In some embodiments, the pharmaceutically acceptable topical formulation includes sorbitol. In particular embodiments, the pharmaceutically acceptable topical formulation includes one or more of sorbitol, glycerin, benzyl alcohol, cetyl alcohol, glyceryl stearate, isopropyl myristate, paraffin, PEG 400, PEG 100 stearate, PEG 4000, petrolatum, peppermint oil, saccharin sodium, and purified water.

In various embodiments of the pharmaceutically acceptable topical formulation, benzocaine is present in an amount from about 2% (w/w) to about 15% (w/w) based on the total weight of the pharmaceutically acceptable topical formulation. In some embodiments, benzocaine is present in an amount from about 5% (w/w) to about 10% (w/w) based on the total weight of the pharmaceutically acceptable topical formulation. In certain embodiments, benzocaine is present in an amount from about 6% (w/w) to about 8% (w/w) based on the total weight of the pharmaceutically acceptable topical formulation. In particular embodiments, benzocaine is present in an amount of about 7% (w/w) based on the total weight of the pharmaceutically acceptable topical formulation.

In various embodiments of the pharmaceutically acceptable topical formulation, capsaicin is present in an amount from about 0.01% (w/w) and about 0.075% (w/w) based on the total weight of the pharmaceutically acceptable topical formulation. In some embodiments, the capsaicin is present in an amount from about 0.014% (w/w) to about 0.018% (w/w) based on the total weight of the pharmaceutically acceptable topical formulation. In particular embodiments, capsaicin is present in an amount about 0.016% (w/w) based on the total weight of the pharmaceutically acceptable topical formulation.

In various embodiments, the pharmaceutically acceptable topical formulation comprises benzocaine in an amount from about 6% (w/w) to about 8% (w/w) and capsaicin in an amount from about 0.014% (w/w) to about 0.018% (w/w) based on the total weight of the pharmaceutically acceptable topical formulation, and a polyethylene glycol and sorbitol. In some embodiments, the pharmaceutically acceptable topical formulation comprises benzocaine in an amount about 7% (w/w) and capsaicin in an amount about 0.016% (w/w) based on the total weight of the pharmaceutically acceptable topical formulation, and a polyethylene glycol and sorbitol. In certain embodiments, the pharmaceutically acceptable topical formulation comprises benzocaine in an amount about 7% (w/w) and capsaicin in an amount about 0.016% (w/w) based on the total weight of the pharmaceutically acceptable topical formulation, and one or more of sorbitol, glycerin, benzyl alcohol, cetyl alcohol, glyceryl stearate, isopropyl myristate, paraffin, PEG 400, PEG 100 stearate, PEG 4000, petrolatum, peppermint oil, saccharin sodium, and purified water. In particular embodiments, the pharmaceutically acceptable topical formulation comprises benzocaine in an amount about 7% (w/w) and capsaicin in an amount about 0.016% (w/w) based on the total weight of the pharmaceutically acceptable topical formulation, and sorbitol, glycerin, benzyl alcohol, cetyl alcohol, glyceryl stearate, isopropyl myristate, paraffin, PEG 400, PEG 100 stearate, PEG 4000, petrolatum, peppermint oil, saccharin sodium, and purified water.

Compositions of the present invention may be administered topically, for example, in the form of a cream, a gel, or a combination thereof.

Creams, as known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally included of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semisolid, suspension-type systems. Single-phase gels contain organic macromolecules distributed substantially uniformly throughout the carrier liquid, which is typically aqueous, but also contains an alcohol and, optionally, an oil. In embodiments, “organic macromolecules,” i.e., gelling agents, are crosslinked acrylic acid polymers such as the “carbomer” family of polymers, e.g., carboxypolyalkylenes. Hydrophilic polymers such as polyethylene oxides, polyoxyethylene-polyoxypropylene copolymers, and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin may also be included. In order to prepare a uniform gel, dispersing agents such as alcohol or glycerin can be added, or the gelling agent can be dispersed by trituration, mechanical mixing, or stirring, or combinations thereof.

In particular embodiments, the pharmaceutically acceptable topical formulation as described herein is in the form of a cream. In some embodiments, the pharmaceutically acceptable topical formulation is in the form of a gel. In various embodiments, the pharmaceutically acceptable topical formulation is in the form of a gel and a cream.

In another aspect, the present teachings provide a method of treating pain in a patient in need thereof, comprising administering topically to the patient a therapeutically effective amount of a pharmaceutically acceptable topical formulation as described herein.

In some embodiments, the pain is chronic or acute. The pain can be back pain, spinal pain, skeletal pain, or joint pain. The pain can be muscle spasm induced pain such as a back muscle spasm induced pain.

In various embodiments, a therapeutically effective amount of a pharmaceutically acceptable topical formulation as described herein can be applied, for example, rubbed on, to the area where pain is being experienced. The effects of the administration can be realized nearly immediately but after a few minutes with the pain subsiding or completely disappearing for up to about 6 hours, about 12 hours, about 18 hours, or about a day. If the pain reoccurs within a day, the pharmaceutically acceptable topical formulation can be reapplied up to two times per day. In addition, a larger amount of the pharmaceutically acceptable topical formulation can be used to provide a therapeutically effective amount of the pharmaceutically acceptable topical formulation.

### EXAMPLES

The following examples are provided for illustrative purposes only, and are not intended to limit the scope of the disclosure.

### Example 1

Mix thoroughly two tubes of Iodent® Maximum Strength Oral Analgesic Gel (Benzocaine 20%) (11.9 g each) with one tube of Sheffield Pharmaceuticals Original Strength Arthritis & Muscle Pain Relief Cream—Thermarub™ Topical Analgesic Cream (0.025% Capsaicin) (43 g), which provides an embodiment of a pharmaceutically acceptable topical formulation of the invention.

Use of an applicator and latex or similar gloves for delivery of a therapeutically effective amount of the formulation to the site of pain is recommended.

### Example 2

An individual was experiencing back pain. A half dollar-size amount of the pharmaceutically acceptable topical formulation of Example 1 was applied to the back of the individual at the site of the back pain. After rubbing in the formulation, the pain was noticeably reduced within minutes, with the pain completely gone after one hour and remaining gone for about a day.

### EQUIVALENTS

The disclosure may be embodied in other specific forms without departing from the spirit or essential characteristics thereof. The foregoing embodiments are therefore to be considered in all respects illustrative rather than limiting the disclosure described herein. Those skilled in the art will recognize, or be able to ascertain using no more than routine experimentation, many equivalents to the specific embodiments of the disclosure described herein. Such equivalents are intended to be encompassed by the following claims. Scope of the invention is thus indicated by the appended claims rather than by the foregoing description, and all changes that come within the meaning and range of equivalency of the claims are intended to be embraced therein.

### INCORPORATION BY REFERENCE

The entire contents of all patents, published patent applications, websites, and other references cited herein are incorporated by reference herein in their entireties.

1. A pharmaceutically acceptable topical formulation comprising:
benzocaine, wherein benzocaine is present in an amount from about 2% (w/w) to about 20% (w/w) based on the total weight of the pharmaceutically acceptable topical formulation; and
capsaicin, wherein the capsaicin is present in an amount from about 0.005% (w/w) to about 0.1% (w/w) based on the total weight of the pharmaceutically acceptable topical formulation,
wherein the pharmaceutically acceptable topical formulation is for topical application.

2. The pharmaceutically acceptable topical formulation of claim 1, further comprising a polyethylene glycol.

3. The pharmaceutically acceptable topical formulation of claim 1, further comprising sorbitol.

4. The pharmaceutically acceptable topical formulation of claim 1, wherein benzocaine is present in an amount from about 5% (w/w) to about 15% (w/w) based on the total weight of the pharmaceutically acceptable topical formulation.

5. The pharmaceutically acceptable topical formulation of claim 1, wherein benzocaine is present in an amount of about 7% (w/w) based on the total weight of the pharmaceutically acceptable topical formulation.

6. The pharmaceutically acceptable topical formulation of claim 1, wherein capsaicin is present in an amount from about 0.01% (w/w) and about 0.075% (w/w) based on the total weight of the pharmaceutically acceptable topical formulation.

7. The pharmaceutically acceptable topical formulation of claim 1, wherein capsaicin is present in an amount of about 0.16% based on the total weight of the pharmaceutically acceptable topical formulation.

8. The pharmaceutically acceptable topical formulation of claim 1, wherein the topical formulation is in the form of a cream.

9. The pharmaceutically acceptable topical formulation of claim 1, wherein the topical formulation is in the form of a gel.

10. The pharmaceutically acceptable topical formulation of claim 1, wherein the topical formulation is in the form of a gel and a cream.

11. The pharmaceutically acceptable topical formulation of claim 2, wherein the polyethylene glycol is one or more of PEG 400, PEG 4000, and PEG-100 stearate.

12. The pharmaceutically acceptable topical formulation of claim 1, wherein the topical formulation comprises PEG 400, PEG 4000, and PEG-100 stearate.

13. The pharmaceutically acceptable topical formulation of claim 1, further comprising one or more of sorbitol, glycerin, benzyl alcohol, cetyl alcohol, glyceryl stearate, isopropyl myristate, paraffin, PEG 400, PEG 100 stearate, PEG 4000, petrolatum, peppermint oil, saccharin sodium, and purified water.

14. The pharmaceutically acceptable topical formulation of claim 1, further comprising sorbitol, glycerin, benzyl alcohol, cetyl alcohol, glyceryl stearate, isopropyl myristate, paraffin, PEG 400, PEG 100 stearate, PEG 4000, petrolatum, peppermint oil, saccharin sodium, and purified water.

15. A method of treating pain in a patient in need thereof, comprising administering topically to the patient a therapeutically effective amount of the pharmaceutically acceptable topical formulation of claim 1.

16. The method of claim 15, wherein the pain is chronic or acute.

17. The method of claim 15, wherein the pain is back pain, spinal pain, or joint pain.

18. The method of claim 15, wherein the pain is muscle spasm induced pain.

19. The method of claim 15, wherein the pain is back muscle spasm induced pain.
